(19) United States
(12) Patent Application Publication
(10) Pub. No.: US 2024/0341646 A1
(43) Pub. Date: Oct. 17, 2024

(54) PHYSIOLOGICAL MEASUREMENT CALIBRATION

(71) Applicant: Masimo Corporation, Irvine, CA (US)

(72) Inventors: Prashanth Iyengar, Irvine, CA (US); Jerome Novak, Aliso Viejo, CA (US); Prashanth Rengaswamy Chandran, Irvine, CA (US)

(21) Appl. No.: 18/750,781

(22) Filed: Jun. 21, 2024

Related U.S. Application Data
(63) Continuation of application No. 17/061,945, filed on Oct. 2, 2020, now Pat. No. 12,042,285.
Application No. 17/061,945 is a continuation of application No. 15/499,806, filed on Apr. 27, 2017, now Pat. No. 10,827,961.
Application No. 15/499,806 is a continuation of application No. 14/160,322, filed on Jan. 21, 2014, now abandoned.
Application No. 14/160,322 is a continuation-in-part of application No. 14/011,567, filed on Aug. 27, 2013, now abandoned.
(60) Provisional application No. 61/694,723, filed on Aug. 29, 2012.
(60) Provisional application No. 61/713,833, filed on Oct. 15, 2012.

(51) Int. Cl. A61B 5/1495; A61B 5/026; A61B 5/145; A61B 5/1455; A61B 5/1491

(52) U.S. Cl. CPC A61B 5/1495; A61B 5/026; A61B 5/14546; A61B 5/1455; A61B 5/1491; A61B 2562/0238; A61B 2562/04

(57) ABSTRACT

The present disclosure provides a calibration system and method for calibrating a physiological measurement based on a variable that affects the measurement. The variable can be a related physiological measurement. The technique can be implemented to obtain robust calibrations with minimal test data and computational effort.

### PRIORITY CLAIM

The present application is a continuation of U.S. patent application Ser. No. 17/061,945, filed Oct. 2, 2020, titled “Physiological Measurement Calibration”, which is a continuation of U.S. patent application Ser. No. “15/499,806, filed Apr. 27, 2017, titled “Physiological Measurement Calibration”, now U.S. Pat. No. 10,827,961, issued Nov. 10, 2020, which is a continuation of U.S. patent application Ser. No. 14/160,322, filed Jan. 21, 2014, titled “Physiological Measurement Calibration”, which is a continuation-in-part of U.S. patent application Ser. No. 14/011,567, filed Aug. 27, 2013, titled “Physiological Measurement Calibration”, which claims priority benefit under 35 U.S.C. § 119 (c) to U.S. Provisional Patent Application Ser. No. 61/694,723, filed Aug. 29, 2012, titled Physiological Measurement Calibration, and U.S. Provisional Patent Application Ser. No. 61/713,833, filed Oct. 15, 2012, titled Physiological Measurement Calibration, each of which is hereby incorporated in its entirety by reference herein.

### RELATED APPLICATIONS

The present Application is related to the following patents and patent applications: U.S. Pat. No. 6,606,511, titled “PULSE OXIMETRY PULSE INDICATOR,” filed on Jan. 6, 2000, and issued on Aug. 12, 2003; U.S. Pat. No. 7,764,982, titled “MULTIPLE WAVELENGTH SENSOR EMITTERS,” filed on Mar. 1, 2006, and issued on Jul. 27, 2010; U.S. Pat. No. 8,130,105, titled “NON-INVASIVE MULTI-PARAMETER PATIENT MONITOR,” filed on Mar. 1, 2006, and issued on Mar. 6, 2012; U.S. patent application Ser. No. 12/011,011, entitled “PERFUSION TREND INDICATOR,” filed on Jan. 22, 2008, and published as U.S. Patent Application Publication No. 2008/0221464 on Sep. 11, 2008; U.S. patent application Ser. No. 12/783,436, entitled “HEMOGLOBIN DISPLAY AND PATIENT TREATMENT,” filed on May 19, 2010, and published as U.S. Patent Application No. 2010/0298675 on Nov. 25, 2010.

The U.S. Patents and Patent Application Publications referenced above are expressly bodily incorporated in their entirety, and are part of the present disclosure. All embodiments described herein are compatible with and/or are components of the embodiments described in the above reference patents and patent applications. Some or all of the features described herein can be used or otherwise combined with any of the features described in the Appendices.

### BACKGROUND

Multiple wavelength optical sensors can be used to non-invasively measure physiological parameters, such as SpO₂, HbCO, HbMet, perfusion index (PI), non-invasive total hemoglobin (SpHb), and other physiological parameters. Noninvasive physiological measurements are often affected by a number of different variables. These variables can include differences in manufacturing tolerances between devices, differences in skin pigmentation and bone structure among patients, operating temperatures, movement, as well as a host of other variables that are often difficult to determine and account for, including, for example, various changes in a patient's physiology that may occur during a measurement.

In some current approaches, a multiple wavelength optical sensor causes light of several wavelengths to be shined into the measurement site of a patient's tissue. The tissue of the patient attenuates the light, which is then detected by a detector. The detected signal is sent to a processor for processing. One drawback of these current measurement systems, among others, is that determining a physiological measurement based on the raw detected signal can be difficult, and often requires a number of different considerations pertaining to other physiological parameters.

### SUMMARY

For purposes of summarizing the disclosure, certain aspects, advantages and novel features of several embodiments have been described herein. It is to be understood that not necessarily all such advantages can be achieved in accordance with any particular embodiment of the embodiments disclosed herein. Thus, the embodiments disclosed herein can be embodied or carried out in a manner that achieves or optimizes one advantage or group of advantages as taught herein without necessarily achieving other advantages as can be taught or suggested herein.

The present disclosure provides a robust calibration technique for physiological measurements of interest. In particular, the present disclosure provides a mapping to calibrate observed measurements so that they are less affected, or entirely unaffected, by variables that can affect the observed measurements of the parameters. For example, in one embodiment, robust non-invasive total hemoglobin (SpHb) measurements are made such that changes in a patient's perfusion (as can be measured by a perfusion index (PI)), do not affect the observed measurements of SpHb.

### BRIEF DESCRIPTION OF THE DRAWINGS

The foregoing aspects and many of the attendant advantages will become more readily appreciated as the same become better understood by reference to the following detailed description when taken in conjunction with the accompanying drawings, wherein:

FIG. 1 is a general block diagram of a physiological measurement system that can be calibrated according to techniques of the present disclosure;

FIG. 2 is a flowchart illustrating an example technique for calibrating a physiological measurement system;

FIG. 3 is a flowchart illustrating an example technique for obtaining measurements of physiological parameters using the physiological measurement system of FIG. 1; and

FIG. 4 is a graph illustrating example mathematical relationships which can be analyzed to implement the example technique shown in FIG. 2.

### DETAILED DESCRIPTION

### Overview of the Present Disclosure

The observation of some physiological measurements can be affected by changes in other physiological parameters. For example, an observed SpHb measurement can be affected by how perfused a patient is. Other variables can also affect an SpHb measurement such as, for example, temperature, desaturation, and other physiological parameters. Of course, it is to be understood that the present disclosure is not limited to the correction of SpHb, but more generally relates to measurement calibration of any physiological parameter that can be affected by a related parameter.

Typically, a vast amount of testing is performed on many different patients in order to determine an calibration equation that will relate the measured data to a correct physiological measurement. It is very difficult to both measure and account for all of the different variables that can affect a particular measurement. For example, while testing a patient for SpHb changes, the patient's perfusion index (PI) can change. Attempting to correlate all of the PI changes and accurately account for them while measuring changes in SpHb is extremely difficult, expensive, and time-consuming, as a large amount of data is often required. Moreover, such inconsistent testing leads to less accurate and precise results, particularly at extreme measurements. Even when a large amount of data is available, it may still be necessary to measure the various variables separately and then adjust or re-compute the desired measurement in real time to account for the variables.

The present disclosure provides a solution that allows for a measurement calibration which requires significantly less data and is much easier to implement than previous methods. Moreover, the present disclosure provides a calibration method that is more robust, particularly at extreme measurements. The present disclosure will be described with respect to correcting SpHb for PI changes, but it is to be understood that the methods described herein can be applied to other measurements as well.

Specifically, the present disclosure provides a technique for isolating changes due to a single variable at a time and then generating a robust calibration for the desired parameter. Any number of variables can be accounted for separately. Accounting for the effects of a single variable at a given time provides the quickest and least intensive solution.

In an embodiment, SpHb measurements are made impervious to interference from unrelated physiological changes. The present disclosure takes advantage of the knowledge that the optics used to obtain the SpHb measurements can be approximated to be linear in the regions of interest. This makes it possible to map ratio measurements into an orthogonal vector, making SpHb measurements uncorrelated with the effects due to PI changes and/or other variables that affect SpHb measurements. This essentially takes away any affect that changes in PI would make on SpHb measurements and thus removes the necessity of separately measuring and accounting for PI and other variables in SpHb measurements during patient measurements. Thus, the SpHb measurements become more robust.

In a particular example, a living patient can be cooled, and then warmed, to measure the effects of PI on SpHb. As the patient's body temperature rises, the effects of PI are measured with respect to internal measurements relevant to SpHb. For example, as the PI increases, the ratios of absorption levels of different wavelengths obtained using the optical measurements are observed to change. These measurements are then used determine a mapping of ratios to SpHb measurements that is less affected by changes in PI while measuring SpHb.

Once the isolated measurements are complete, for example, once a sufficient amount of data is obtained through PI tests while holding other variables constant, the data is used to model the relationship between PI and some of the internal measurements that in turn affect the prediction of the desired parameter, for example SpHb. This relationship can be used to adjust the measurement of the desired parameter. This calibration information can further be used to calibrate a physiological measurement system to obtain measurements for SpHb that are decorrelated from other variables.

### Example Physiological Measurement System

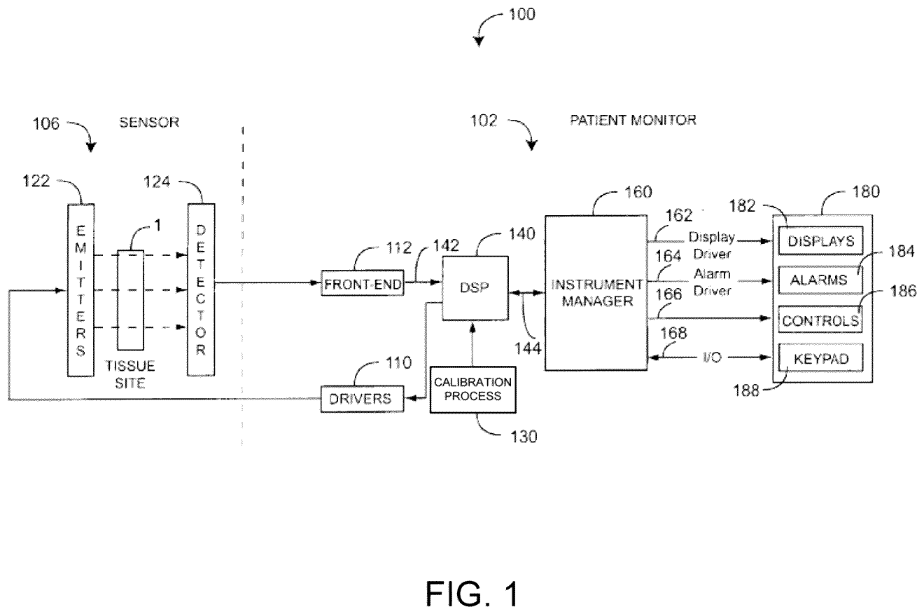

FIG. 1 illustrates an embodiment of a physiological measurement system 100. The physiological measurement system 100 can measure various physiological parameters, such as SpO₂, desaturation, pulse rate (PR), perfusion index (PI), total hemoglobin (SpHb), HbCO, and HbMet. Further, the physiological measurement system 100 can calculate and display values of physiological parameters, as well as fluctuations, trends, or other indications of changes in the physiological parameters measured by the physiological management system 100. In some embodiments, the physiological measurement system 100 can provide displays, alarms, or controls responsive to physiological parameters (such as SpHb, PI, or other parameters) or changes thereto that can affect the treatment of the patient whose physiological parameters are being measured.

As shown in FIG. 1, the physiological measurement system 100 includes a patient monitor 102 and a sensor 106. The sensor 106 attaches to a patient's tissue site 1 and includes an array of one or more emitters 122 capable of irradiating the tissue site 1 with at least two known wavelengths of light, such as red and infrared (IR) wavelengths commonly utilized in pulse oximeters. The emitters 122 can be configured to emit other wavelengths as well, and these wavelengths can be emitted in addition to or instead of the red and IR wavelengths discussed above. For example, the emitters 122 can be configured to emit at least three known wavelengths of light, which can include a red wavelength, an IR wavelength, and a third wavelength. The third wavelength can itself be a red or IR wavelength, or can be any other wavelength selected as desired. In some embodiments, each individual emitter of the emitters 122 is configured to emit a unique wavelength. In other embodiments, an emitter of the emitters 122 can be configured to emit multiple wavelengths, either simultaneously or one at a time. In some embodiments, the emitters 122 are arranged in rows and/or columns of individual emitters, with each individual emitter being configured to emit light in response to an electrical drive signal. Further information pertaining to the emitters 122 can be found in U.S. Pat. No. 7,764,982, titled “MULTIPLE WAVELENGTH SENSOR EMITTERS,” filed on Mar. 1, 2006, and issued on Jul. 27, 2010. The disclosure of this patent was previously incorporated by reference in its entirety.

The light emitted by the emitters 122 is attenuated by the tissue site 1 as it passes through to the one or more detectors 124, which can detect the light emitted by the emitters 122 and attenuated by the tissue site 1. In some embodiments, an individual detector is provided for each different wavelength emitted by the emitters 122. For example, if the emitters 122 are configured to emit a red wavelength, an IR wavelength, and a third wavelength, three detectors can be provided: one to detect the red wavelength, one to detect the IR wavelength, and one to detect the third wavelength. In other embodiments, a single detector of the detectors 124 can detect multiple wavelengths of light.

With continued reference to FIG. 1, the patient monitor 102 communicates with the sensor 106 to receive one or more intensity signals indicative of one or more physiological parameters and displays the parameter values. The drivers 110 can convert digital control signals into analog drive signals capable of driving sensor emitters 122. A front-end 112 can convert composite analog intensity signal(s) from light sensitive detector(s) 124 into digital data 142 to be provided to the digital signal processor (DSP) 140. The digital data 142 includes channels corresponding to each emitter wavelength, such as a red channel, an IR channel, and a channel for a third wavelength. The digital data 142 can be representative of a change in the absorption of particular wavelengths of light as a function of the changes in body tissue resulting from pulsing blood and/or from changing amounts of hemoglobin in the blood.

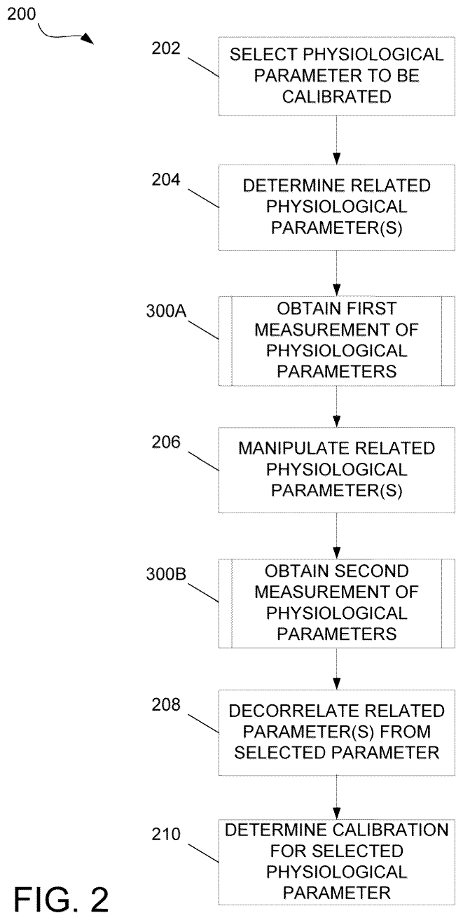

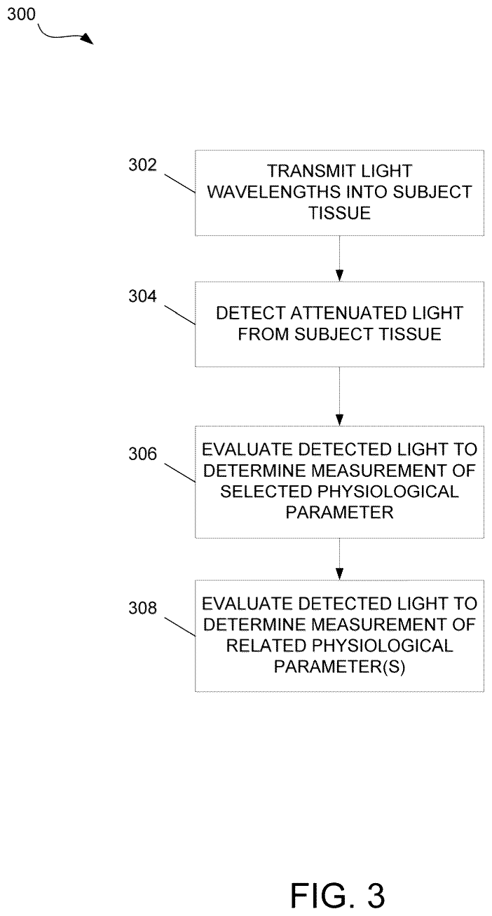

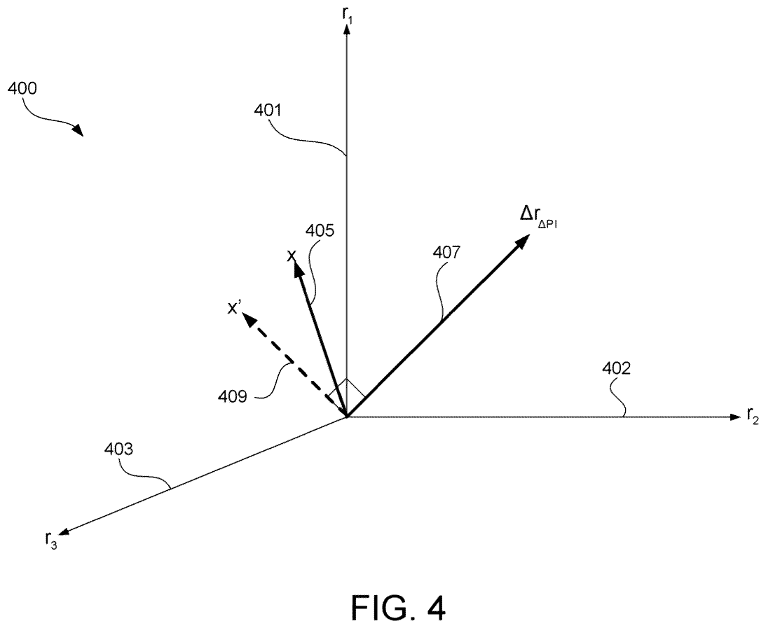

The DSP 140 can include a wide variety of data and/or signal processors capable of executing programs for determining physiological parameters from input data. In an embodiment, the calibration processes 130 can be implemented as a computer system that includes software, firmware, or other form of code or instructions, or logic or other hardware, or a combination of the above. In some embodiments, the DSP 140 can partially or wholly implement one or more calibration processes 130, as described further with respect to FIG. 2, FIG. 3, and FIG. 4. The DSP 140 can, for example, be configured to determine calibration information, and can further use the calibration information to calibrate the sensor 106 to obtain a decorrelated measurement of the selected physiological parameter.

In other embodiments, the calibration processes 130 are implemented as a computer system that is separate from the physiological measurement system 100, as also described further with respect to FIG. 2, FIG. 3, and FIG. 4. For example, the computer system can be provided with data corresponding to a plurality of physiological measurements, without necessarily being capable of taking physiological measurements of a living patient. The computer system can determine calibration information from the plurality of measurements, and provide the calibration information to a physiological measurement system 100 that includes the DSP 140. The DSP 140 can use the provided calibration information to calibrate the sensor 106, substantially as discussed above.

Further shown in FIG. 1, the instrument manager 160 can comprise one or more microcontrollers controlling system management, such as monitoring the activity of the DSP 140. One or more output devices 180 include displays 182, alarms 184 and controls 186. The displays 182 can present information regarding one or more physiological parameter measurements in any of a variety of ways. For example, the displays 182 can present numerical information, such as readouts; or can present graphical information, such as trends and bar graphs. The displays 182 can include one or more LED screens, LCD screens, CRT displays, dot matrix displays, touchscreens, or any other kind of display hardware known in the art. The displays 182 can also include LEDs of various colors that can signify the magnitude of a physiological parameter measurement. Alarms 184 can be visual or audible alerts that can indicate any of a variety of conditions that can be pertinent to a patient's physiological parameters. For example, the alarms 184 can indicate that a particular physiological parameter is, for example, above or below a threshold. The alarms 184 can also indicate that a particular physiological parameter has changed or is trending in a direction that is pertinent to a patient's care. Controls 186 can be inputs to medical equipment, such as drug administration devices, ventilators and fluid IVs, so as to control the amount of administered drugs, ventilator settings or the amount of fluids infused to a patient. The instrument manager 160 can also include an input/output (I/O) port 168 that provides a user and/or device interface for communicating with the monitor 102. User input devices 188 can include a keypad, touch screen, pointing device, voice recognition device, network and computer, among other examples. In an embodiment, the I/O port 168 provides initialization settings for physiological measurement calibrations, as described below. The monitor 102 can also store or display historical or trending data related to SpHb, PI, and other measured parameters or combinations of measured parameters.

### Example Calibration Technique

FIG. 2 depicts an example technique 200 by which a calibration can be determined for a physiological parameter measurement. This example technique 200 can be implemented to calibrate the physiological measurement system 100 of FIG. 1 to obtain a robust measurement, such that the measurement of a selected physiological parameter is not affected by a measurement of related physiological parameter(s). Other physiological measurement systems can also be calibrated using the technique described herein.

At block 202, a physiological parameter for which measurements are to be calibrated is selected. Generally described, a physiological parameter can include any quantifiable aspect of a living patient that can be measured. The selection of the physiological parameter can vary based on the capabilities of the physiological measurement system. For illustrative purposes, specific reference is made herein to calibrating a physiological measurement system to facilitate obtaining a robust SpHb measurement. However, calibrations can be determined for other physiological parameters as well, such that robust measurements of those parameters can be obtained. Such physiological parameters can include, for example, SpO₂, desaturation, pulse rate (PR), perfusion index (PI), HbCO, and HbMet. Calibrations can be determined for still other selected physiological parameters.

At block 204, one or more physiological parameters related to the physiological parameter selected in block 202 are determined. A related physiological parameter can include any parameter that affects the observation of the selected physiological parameter, such that a change in the related physiological parameter can cause a change in the observation of the selected physiological parameter, or vice versa. In a particular example provided for illustrative purposes, if a living patient's SpHb is the physiological parameter selected in block 202, a related physiological parameter can be the living patient's perfusion index (PI). In another embodiment, if the living patient's SpHb is the physiological parameter selected in block 202, the related physiological parameter can be the living patient's desaturation. Still other related parameters are possible. Additionally, in some embodiments, multiple physiological parameters are related to the selected physiological parameter. Returning to the previous example, if the living patient's SpHb is the physiological parameter selected in block 202, both desaturation and PI can be related physiological parameters.

At block 300A, a first measurement is obtained of the selected physiological parameter and of the one or more related physiological parameters. This measurement can be obtained, for example, by the sensor 106, and processed by the DSP 140 as shown in the physiological management system 100 of FIG. 1. An example technique 300 for obtaining a physiological measurement is shown in FIG. 3. Other techniques for obtaining a measurement of physiological parameters are possible.

At block 206, the one or more related physiological parameters are manipulated. In some embodiments, only one related physiological parameter is manipulated. In embodiments in which multiple related physiological parameters have been identified in block 204, one, some, or all of the related physiological parameters can be manipulated. The selected physiological parameter and other related physiological parameters can be held constant while a particular related physiological parameter is manipulated.

In a particular, non-limiting, example, the selected physiological parameter can be a non-invasive total hemoglobin parameter (SpHb) of a living patient, while the related physiological parameter can be a perfusion index (PI) of the living patient. Accordingly, the PI of the living patient can be manipulated in block 206. In an embodiment, during the measurement process, SpHb and all other parameters and variables are held constant while PI is manipulated. PI, in particular, is very difficult to manipulate as it relates to how constricted a patient's blood vessels are. In an embodiment, PI is manipulated by lowering the whole body temperature and then slowly raising the test patient's body temperature. As the patient's body temperature rises, the PI will increase. This is because when the patient is cold, the natural body reaction is to constrain blood vessels in order to keep blood in the body's core and thus keep the vital organs warm. As the body temperature increases, the blood vessels open up and allow more blood to flow in the extremities, thus increasing PI.

Accordingly, in an embodiment, a patient's temperature is lowered using alcohol sprayed on a patient's foot or feet. In an embodiment, the patient is placed in an environment (such as a room) having a particular known temperature. For example, the patient can be placed in an environment that is at room temperature. The patient may then be warmed up. In another example, the patient can be placed in an environment having a relatively cool temperature and allowed to acclimatize to the relatively cool temperature. In an embodiment, the patient's body temperature is increased by applying a body warmer to the patient. A body warmer can include a body blanket warmer, electric blanket, blankets, surgical warming blankets, whole body warmers, heat lamps, space warmers, or any other system for warming the patient.

Of course, other physiological parameters related to SpHb can be individually manipulated as is well known in the art. For example, similar isolated measurements can be made to account for desaturations. This can be done for example, by having a patient hold his or her breath. Other variables can also be isolated and manipulated as well.

At block 300B, a second measurement is obtained of the selected physiological parameter and of the one or more related physiological parameters. This measurement can be obtained, for example, by the sensor 106, and processed by the DSP 140 as shown in the physiological management system 100 of FIG. 1. Again, an example technique 300 for obtaining a physiological measurement is shown in FIG. 3. Other techniques for obtaining a measurement of physiological parameters are possible. Further, the first measurement obtained at block 300A and the second measurement obtained at block 300B can be obtained by the same technique or by different techniques.

In block 208, the selected physiological parameter and the one or more related physiological parameters are decorrelated from one another. In one embodiment, decorrelating the selected physiological parameter and the one or more related physiological parameters includes determining a vector for the selected physiological parameter that is orthogonal to one or more vectors for the one or more related physiological parameters, as discussed further herein with respect to FIG. 4. A calibration function can be minimized to identify the vector. In some embodiments, the calibration function is based on one or more weights assigned to ratios of wavelengths of attenuated light detected by the sensor 106 of the physiological measurement system 100, and thus the vector can include the weights of the ratios. If multiple related physiological parameters are to be decorrelated from the selected physiological parameter, a vector for the selected physiological parameter can be determined that is mutually orthogonal with respect to each of the vectors of the related parameters. Once the vector for the selected physiological parameter is determined, it can be included in calibration information to facilitate calibrating a sensor 106, such that the calibrated sensor is configured to obtain a decorrelated measurement.

Finally, in block 210, a calibration can be determined for the selected physiological parameter, based on the decorrelation performed in block 208. The calibration can be used to calibrate a physiological measurement system 100 to obtain a decorrelated measurement of the selected physiological parameter that reduces or eliminates the effects of the one or more related physiological parameters. In some embodiments, this calibration can be provided as calibration information, which can be processed by the DSP 140 of the physiological measurement system to enable the sensor 106 to obtain a decorrelated measurement of the selected physiological parameter.

Many variations of the example technique 200 are possible. In one embodiment, the related physiological parameter(s) are manipulated more than twice, and thus more than two measurements can be taken and used to decorrelate the selected physiological parameter and the selected physiological parameters. Further, measurements need not be taken from the same living patient. Measurements from different living patients can be obtained, without necessarily manipulating the physiological parameters of the different living patients. Still other variations on the calibration technique 200 are possible.

### Example Physiological Parameter Measurement Technique

FIG. 3 depicts an example technique 300 for obtaining a physiological parameter measurement from a living patient. This example technique 300 can be implemented with the physiological measurement system 100 of FIG. 1, though other physiological measurement systems can be used to obtain physiological parameter measurements as well. Likewise, techniques other than the technique 300 illustrated in FIG. 3 can be used to obtain a physiological parameter measurement. The technique for obtaining a physiological parameter measurement can vary based on the parameter to be measured.

At block 302, light is emitted by the emitters 122, such that the emitted light impinges upon a living patient's tissue at the tissue site 1. As discussed above, in some embodiments, the light wavelengths comprise at least two wavelengths of light, which can include light of a red wavelength and light of an infrared wavelength. In some embodiments, the light wavelengths comprise at least three wavelengths of light, which can include light of a red wavelength, light of an infrared wavelength, and light of a third wavelength, which can be selected as desired.

The tissue at the tissue site 1 attenuates the light emitted by the emitters 122. Thus, in block 304, the attenuated light is detected by the detectors 124 after it emerges from the tissue at tissue site 1. The intensity of each wavelength of light can be determined by the detectors 124 and converted into digital data for processing by the digital signal processor 140, as discussed above with respect to FIG. 1.

At block 306, the detected light can be evaluated to determine a measurement (e.g., a measured value) of the selected physiological parameter. In some embodiments, the selected physiological parameter is a non-invasive total hemoglobin parameter (SpHb) of the living patient. Since hemoglobin causes different degrees of attenuation for different wavelengths of light, the degree to which each wavelength of light is attenuated can be evaluated to determine the SpHb measurement. For example, if three wavelengths of light are emitted by the emitters 122, the attenuation ratio (e.g., the intensity of the attenuated light detected by the detectors 124 divided by the intensity of the unattenuated light emitted by the emitters 122) for each wavelength can be used to determine the SpHb measurement. For example, each wavelength's attenuation ratio can be weighted, and the weighted ratios can be summed to determine the SpHb measurement. Still other techniques for determining an SpHb measurement based on the detected light are possible.

At block 308, the detected light is evaluated to determine a measurement (e.g., a measured value) of one or more related physiological parameters. In some embodiments, the one or more physiological parameters include a perfusion index (PI) of the living patient. More information pertaining to the measurement of PI can be found in U.S. patent application Ser. No. 12/011,011, entitled “PERFUSION TREND INDICATOR,” filed on Jan. 22, 2008, and published as U.S. Patent Application Publication No. 2008/0221464 on Sep. 11, 2008. The disclosure of this application was also previously incorporated by reference above and is also provided as an appendix to this application.

Other parameters can be included in the one or more physiological parameters to be measured. For example, desaturation, pulse rate, tissue temperature, HbMet, HbCO, SpO₂, and the like can be measured as desired. More information on determining physiological parameters can be found at least in U.S. Pat. No. 8,130,105, titled “NON-INVASIVE MULTI-PARAMETER PATIENT MONITOR,” filed on Mar. 1, 2006, and issued on Mar. 6, 2012. The disclosure of this patent was previously incorporated by reference in its entirety.

### Example Decorrelation

Turning now to FIG. 4, a graph 400 is shown to illustrate example mathematical relationships of the present disclosure. The graph has axes r₁ 401, r₂ 402, and r₃ 403. The coordinates r₁, r₂ and r₃ represent ratios of wavelengths obtained from a noninvasive optical sensor 106 having at least three wavelengths, substantially as discussed above with respect to FIG. 1. Of course, more ratios and more wavelengths can be used and an n-dimensional graph can be generated accordingly. Each axis corresponds to a linear calibration fit for SpHb. That is, SpHb is found using the following equation:

$$SpHb = {x}_{1} {r}_{1} + {x}_{2} {r}_{2} + {x}_{3} {r}_{3} \qquad ( Eq . 1 )$$

where x₁, x₂ and x₃ are weights. The original calibration fit for SpHb is illustrated by the vector 405, labeled as X.

The data obtained by isolating and manipulating PI is also illustrated in graph 400 as vector 407, labeled as Δr_{ΔPI}, or in other words, the change in the ratios based on the change in PI. The calibration of SpHb based on PI can be found by determining a set of x values (weights) that minimizes a calibration function based on the first measurement and the second measurement of SpHb. In one embodiment, the calibration function is given by

minf (x) =|RX-t|2 +a|CX|2

where

$${}_{min} f ( x ) = {‖ RX - t ‖}^{2} + α {‖ CX ‖}^{2} \qquad ( Eq . 2 )$$

$$where$$

$$R = [ \begin{matrix}{r}_{{1}_{measurement \cdot 1}} & {r}_{{2}_{measurement \cdot 1}} & {r}_{{3}_{measurement 1}} \\ {r}_{{1}_{measurement 2}} & {r}_{2 measurement 2} & {r}_{{3}_{measurement 2}}\end{matrix} ] \qquad ( Eq . 3 )$$

$$X = [ \begin{matrix}{x}_{1} \\ {x}_{2} \\ {x}_{3}\end{matrix} ] \qquad ( Eq . 4 )$$

$$C = [ \begin{matrix}Δ {r}_{1 Δ PI} & Δ {r}_{2 Δ PI} & Δ {r}_{3 Δ PI}\end{matrix} ] \qquad ( Eq . 5 )$$

$$t = [ \begin{matrix}{tHb}_{1} \\ {tHb}_{2}\end{matrix} ] \qquad ( Eq . 6 )$$

and where tHb₁ and tHb₂ are reference total hemoglobin measurements. For example, tHb₁ and tHb₂ can be obtained by drawing a sample of the patient's blood and using accepted or “gold standard” measurement methods to determine a total hemoglobin value as known in the art. In particular, non-limiting examples, the tHb measurements can be obtained via spectrophotometric analysis or conductometric analysis of the patient's blood sample. The constant α is a non-zero positive real number, which can be selected as desired. A higher a may provide greater decorrelation, but may introduce measurement errors. A lower α may provide a lower measurement error, but may also increase the correlation between SpHb and PI.

Once a set of x values are determined that minimizes the calibration function f(x) in Eq. 1, they can be plotted as vector 409, labeled as X′. The set of X′ values found using Eq. 1 is then used to not only predict SpHb, but also make it insensitive to changes in PI. This vector can be included in calibration information and used to calibrate a sensor 106 and/or DSP 140 of a physiological measurement system 100 to obtain decorrelated measurements of SpHb.

This same process can also be applied to any related physiological parameters that can affect a measurement of a selected physiological parameter. Moreover, multiple related physiological parameters can be decorrelated from the selected physiological parameter. For example, SpHb can be decorrelated from both PI and desaturation. Once sufficient data is obtained, the x values can again be mapped to a new orthogonal vector or taken into account in Eq. 1 to find a mutually orthogonal vector to both the effects of PI and desaturation. Such a process can be applied to any number of variables or to other desired parameters.

Additionally, it will be recognized that other techniques can be used to decorrelate the selected physiological parameter from its related physiological parameters. For example, multiple regression analysis, conjoint analysis, and other techniques can be used to determine the effects of related physiological parameters on the selected physiological parameter, and to generate calibration information such that a decorrelated measurement of the selected physiological parameter can be obtained. Still other techniques are possible.

### Terminology

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor can also include primarily analog components. For example, any of the signal processing algorithms described herein can be implemented in analog circuitry. A computing environment can include any type of computer system, including, but not limited to, a computer system based on a microprocessor, a mainframe computer, a digital signal processor, a portable computing device, a personal organizer, a device controller, and a computational engine within an appliance, to name a few.

The steps of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the inventions described herein can be embodied within a form that does not provide all of the features and benefits set forth herein, as some features can be used or practiced separately from others.

What is claimed is:

1. A physiological monitoring system comprising:
a sensor including a plurality of light emitters configured to shine light of known wavelengths into tissue of a patient and at least one detector configured to detect the light after the light has been attenuated by the tissue; and
a processor in communication with the sensor, the processor configured to receive the detected light and determine a non-invasive total hemoglobin measurement (SpHb) based on calibration information which decorrelates SpHb measurements from changes in the patient's perfusion.

2. The physiological monitoring system of claim 1, wherein the calibration information is determined based on an empirical data.

3. The physiological monitoring system of claim 2, wherein the empirical data is obtained from a cross-section of patients.

4. The physiological monitoring system of claim 3, wherein the perfusion index of the cross section of patients is manipulated by warming the patients.

5. The physiological monitoring system of claim 3, wherein the perfusion index of the cross section of patients is manipulated by cooling the patients.

6. A method for physiological parameter measurement calibration, the method comprising:
shining light of a plurality of wavelengths into a patient's tissue;
detecting the light after the light has been attenuated by the patient's tissue;
obtaining a first measurement of the patient's SpHb and a related physiological parameter, wherein the first measurement is based at least in part on one or more attenuation ratios of absorption energies associated with each known wavelength of the light;
obtaining a second measurement of the patient's SpHb and the related physiological parameter, wherein the patient's SpHb measurement is substantially the same and wherein the related physiological parameter is manipulated and the patient's SpHb is held the same;
combining the first measurement and the second measurement with similar measurements from other patients to determine calibration information useful for decorrelating SpHb measurements from related physiological parameters; and
based on the calibration information, configuring a physiological parameter measurement system to obtain a decorrelated SpHb measurement.

7. The method of claim 6, wherein the related physiological parameter is the patient's perfusion index.

8. The method of claim 7, wherein the patient's perfusion index is manipulated by manipulating the patient's body temperature.

9. The method of claim 8, wherein the patient's body temperature is manipulated by spraying alcohol on the patient.

10. The method of claim 8, wherein the patient's body temperature is manipulated by a body warmer applied to the patient.

11. The method of claim 9, wherein the patient's body temperature is manipulated by placing the patient in an environment having a known temperature.

12. The method of claim 6, wherein the related physiological parameter is the patient's desaturation.

13. The method of claim 12, wherein the patient's desaturation is manipulated by the patient holding his or her breath.

14. Non-transitory computer storage having stored thereon a system configured to execute in one or more processors, the system comprising a calibration module configured to:
obtain a plurality of correlated measurements, each correlated measurement comprising a measurement of a selected physiological parameter and a measurement of a related physiological parameter;
based on the plurality of correlated measurements, decorrelate the selected physiological parameter and a first related physiological parameter; and
based on the decorrelation of the selected physiological parameter and the related physiological parameter, determine calibration information;
wherein the calibration information facilitates obtaining a decorrelated measurement of the selected physiological parameter.

15. The non-transitory computer storage of claim 14, wherein the calibration module is further configured to use the calibration information to calibrate a physiological parameter measurement system to obtain the decorrelated measurement of the selected physiological parameter.

16. The non-transitory computer storage of claim 14, wherein the calibration module is configured to decorrelate the selected physiological parameter and the first related physiological parameter by minimizing a value of a correlation function that relates the selected physiological parameter and the first related physiological parameter.

17. The non-transitory computer storage of claim 14, wherein:
each of the plurality of correlated measurements further comprises a measurement of a second related physiological parameter; and
the calibration module is further configured to decorrelate the selected physiological parameter, the first related physiological parameter, and the second related physiological parameter.

18. The non-transitory computer storage of claim 17, wherein the calibration module is configured to decorrelate the selected physiological parameter, the first related physiological parameter, and the second related physiological parameter by:
determining a first vector that represents a change in the selected physiological parameter with respect to a change in the first related physiological parameter;
determining a second vector that represents a change in the selected physiological parameter with respect to a change in the second related physiological parameter; and
determining a third vector that is orthogonal to both the first vector and the second vector;
wherein the calibration information comprises the third vector.

19. The non-transitory computer storage of claim 17, wherein:
the selected physiological parameter is SpHb;
the first related physiological parameter is perfusion index; and
the second related physiological parameter is desaturation.
